(19) United States
(12) Patent Application Publication
(10) Pub. No.: US 2007/0016108 A1
(43) Pub. Date: Jan. 18, 2007

(54) Method for 3D visualization of vascular inserts in the human body using the C-arm

(75) Inventors: Estelle Camus, Erlangen (DE); Volker Heer, Gundelsheim (DE); Markus Lendl, Ottensoos (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS AKTIENGESELLSCHAFT

(21) Appl. No.: 11/486,697

(22) Filed: Jul. 14, 2006

(30) Foreign Application Priority Data
Jul. 14, 2005 (DE) 10 2005 032 974.8

(51) Int. Cl. A61B 5/107

(52) U.S. Cl. 600/587

(57) ABSTRACT

The invention relates to a method for 3D visualization of vascular inserts in the human body using C-arm radiography comprising: firstly recording a contrast-agent-free vessel system having a vascular insert of two series x-ray recordings with different angulations, secondly contrast-agent-based recording the same vessel system of two series x-ray recordings, processing the first two series recordings for improving image quality of the image data sets containing the insert representation, matching the vessel anatomy from the second two series recordings to the insert representation contained in the processed first two series recordings by 2D matching, computing a 3D data set of a region around the insert enclosing the insert as completely as possible based on the processed first two series recordings, computing a 3D data set of the vessel system based on the second two series recordings, and superimposing the two computed 3D data sets based on the 2D matching.

### CROSS REFERENCE TO RELATED APPLICATIONS

This application claims priority of German application No. 10 2005 032 974.8 filed Jul. 14, 2005, which is incorporated by reference herein in its entirety.

### FIELD OF THE INVENTION

The present invention relates to a method for 3D visualization of vascular inserts such as stents or heart valves in x-ray recordings. The present invention specifically relates to high-resolution 3D representations of such inserts in vessel anatomies affected by respiration or cardiac movement.

### BACKGROUND OF THE INVENTION

Intravascular interventions are increasingly based on introducing one or more inserts (e.g. stents, artificial heart valves, etc.) into the pathogenic sections of a vessel, e.g. into the narrowed region of a stenotized vessel.

The present invention will be described, without limiting its generality, using the example of stent implantation in the coronary vessel region, the visualization of which constitutes a challenge which has so far been only inadequately met because of rapid cardiac movement.

The problem from a medical engineering viewpoint is that, after a stent implantation, on the one hand the structure of the stent (radially expandable reticulated tube) and therefore its expanded state cannot be satisfactorily represented. Also its position relative to the vessel or more precisely the vascular tree and/or to other anatomical structures can currently only be imaged inadequately on x-ray projection images.

This is because the stent structures are very fine, the coronary vessels (and therefore the stent) are constantly moved by heart beat and respiration and the time and spatial resolution of current x-ray systems are insufficient to make the stent clearly visible and show it without artifacts. Moreover, a two-dimensional x-ray projection image (e.g. an x-ray C-arm image) is inadequate in order to be able to determine or represent the exact position and attitude of a stent as a three-dimensional object in a three-dimensional vascular tree.

Numerous methods have been developed specifically for improving the visualization of stents (see e.g. U.S. Pat. No. 5,457,728 and U.S. Pat. No. 5,054,045). With these methods, a plurality of x-ray images of a stent are recorded at a precisely defined projection, the region of interest (ROI) relevant for the stent is selected and the image data of said ROI is summed. In this way the signal-to-noise ratio in the ROI is improved, thereby increasing the detectability of the stent or rather the resolution of its fine structure. Image summing thus increases the signal contrast while at the same time reducing the uncorrelated noise by means of averaging.

However, inherent constraints of these methods result in the serious disadvantage that only the inserts of interest themselves (stents, heart valve, etc.) can be represented, but not the surrounding vessel anatomy, as object representation does not permit the use of contrast agents.

Moreover, these prior art methods only permit two-dimensional representations of the two three-dimensional objects of interest, i.e. stent and vessel anatomy.

### SUMMARY OF THE INVENTION

This object of the present invention is therefore to further improve the representation of vascular inserts together with the surrounding vascular anatomy.

This object is achieved according to the invention by the features set forth in the independent claims. The dependent claims further develop the central idea of the invention in a particularly advantageous manner.

There is claimed a method for 3D visualization of vascular inserts in the human body using C-arm x-ray radiography, comprising the following steps:
S1: Recording of a contrast-agent-free vessel system having a vascular insert, in the form of two series of x-ray recordings with different angulations,
S2: Contrast-agent-based recording of the same vessel system having a vascular insert, in the form of two series of x-ray recordings using the same equipment configuration as in step S1,
S3: Processing of the two series of x-ray recordings according to step S1 by means of image processing methods for enhancing the image quality of the image data sets containing the insert representation,
S4: Matching of the vessel anatomy from step S2 to the insert representation from step S3 by means of 2D matching of the insert representations of the series according to step S3 to the series according to step S2,
S5: Computation of a 3D data set of a region around the insert which encloses the insert as completely as possible, on the basis of the enhanced image data set computed in step S3,
S6: Computation of a 3D data set of the vessel system on the basis of the data set of step S2 and
S7: Superimposition of the 3D data set calculated in step S6 on the 3D data set computed in step S5, on the basis of the 2D matching according to step S4.

This method can be refined according to the invention by preceding step S1 with a step S1A of contrast-agent-based recording of the vessel system using at least two different x-ray projections in respect of the projection angle, this being followed by a step S1B in which a 3D data set is computed on the basis of the recordings obtained in S1A, and step S6 being followed by a step S6A in which 3D registration of the 3D data set computed according to S6 with the 3D data set computed according to S1B takes place, and step S7 being replaced by step S7A in which the 3D data set computed in S5 is superimposed on the 3D data set computed in S1B on the basis of the 2D matching according to S4 and on the basis of the 3D registration according to S6A.

In the case of refining the method according to the invention, a pre-operatively present 3D data set from previous 3D CT imaging or 3D MRT imaging can be used as an alternative to steps S1A und S1B.

It is likewise advantageous if the angulation difference is 90°.

The image processing methods in step S3 are advantageously based on segmentation or selection techniques.

According to the invention, the vascular insert can be a stent, a bypass or an artificial heart valve.

Likewise, in a further embodiment of the invention the recording of the vessel system in steps S1 and S2 can advantageously be triggered by an ECG or a blood pressure measurement.

Also advantageous is the matching in step S4 using ECG and respiration signals recorded synchronously to the image signals. This facilitates the search for corresponding image pairs from steps S1 und S2 having similar cardiac phase and lung states.

In addition, a position sensor on or in the insert can be advantageously used for the registering of the 3D data sets according to step S6A.

An apparatus for carrying out the method according to one of the preceding claims is also claimed according to the invention.

### BRIEF DESCRIPTION OF THE DRAWINGS

Further advantages, features and characteristics of the present invention will now be explained in greater detail using examples with reference to the accompanying drawings in which:

FIG. 1 shows a flowchart of the method according to the invention, and

FIG. 2 shows a flowchart of a variant of the method according to the invention.

### DETAILED DESCRIPTION OF THE INVENTION

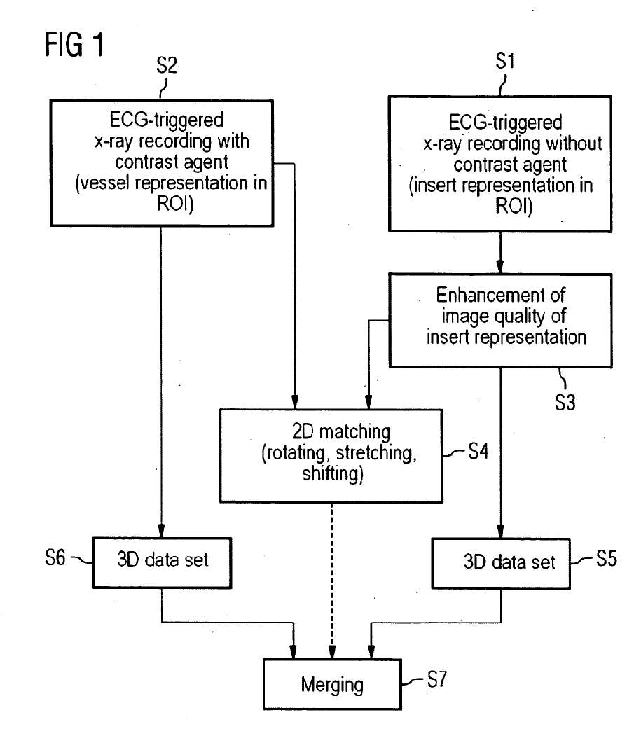

FIG. 1 illustrates the method according to the invention in the form of a method flowchart. The method provides, on the one hand, improved 3D visualization of the vascular insert by means of inventive image post-processing and, on the other hand, improved three-dimensional visualization of the vessel anatomy in which the vascular insert is embedded. This method is based in principle on the creation of two optimized 3D data sets which are finally merged after 2D matching, the image data of one 3D data set (the vessel anatomy) being obtained from contrast agent recordings, whereas for the other 3D data set (improved visualization of the vascular insert) recording is performed without contrast agent.

The individual steps of the method will now be explained:

In step S1, ECG-triggered recording of a narrow region of interest (ROI) around the vascular insert takes place. This is x-ray-based recording without contrast agent in order, on the one hand, primarily to enable the insert to be visualized and, on the other hand, to enable image processing to be performed which will improve the visibility of the insert. The ECG data is used solely to enable the images which are to be recorded in step S1 to be captured in the same phase of cardiac rhythm as in the following step S2. For only then can the 3D data sets of vessel anatomy and insert be usefully merged. It should be noted that, as an alternative to ECG data, blood pressure data for the same vascular region can also be used and thus ECG triggering may be replaced by blood pressure triggering.

In step S2, ECG-triggered recording of the same vessel system having the vascular insert as in step S1 is performed and with the same configuration of the recording equipment (e.g. identical C-arm angulation and position). X-ray projections in step S2 must include the insert, even if it is poorly visible because of the use of contrast agent.

The x-ray recordings of steps S1 and S2 are made in at least two planes in the form of at least two series of x-ray photographs, the at least two series being significantly different in respect of C-arm angulation (the optimum angulation difference would be approximately 90° in the case of two series).

The two angiographic image data sets with (S2) and without (S1) contrast agent are used in order to be able to better computationally compensate possible movements of the object of interest (displacement and rotation of the insert e.g. due to cardiac movement or patient movement) between two 3D data sets computed from the two steps S1 und S2.

However, the step of computing the 3D data set from the x-ray recording without contrast agent (S1) is preceded by a step S3 in which the image quality of the insert representation is first improved. The improvement is effected by image processing of the series of x-ray recordings acquired according to step S1 using known segmentation or selection methods (e.g. on the basis of noise reduction, edge enhancement, etc.) and is carried out in order to maximize the signal-to-noise ratio in the immediate vicinity of the insert and therefore increase the visibility of said insert.

Likewise, still before 3D data set computation (S5), in a fourth step S4 the vessel anatomy images from step S2 are matched to the insert representation images from step S1 by 2D matching (scaling, rotating, stretching, shifting, elastic deformation of the images so that the combination of vessel anatomy and insert corresponds exactly to reality). 2D matching is carried out in order to compensate possible displacements or dilations of the vessel containing the insert due to respiration or slight movement of the patient between the recordings of steps S1 and S2. This can be done automatically (e.g. on the basis of anatomical or artificial landmarks) or manually. The position and angulation, assumed to be the same, of the radioscopy equipment and the cardiac movement and respiratory phase ascertainable from ECG and respiration signals can be used as the starting point for matching.

In the fifth step S5 already numerously mentioned there is computed, on the basis of the improved data set computed in step S3 , a 3D data set which constitutes a 3D model of the region of interest (ROI) of the insert. In this 3D model (3D data set) the insert is essentially clearly visible.

In a sixth step S6 a 3D data set is likewise computed, but on the basis of the series of images recorded in step S3 which, because of the contrast agent, result in a markedly high-resolution 3D model of the vessel system (e.g. a pathogenic coronary vessel) containing the ROI of the insert. The insert is difficult to detect in this contrast-agent-based 3D model of the vessel system.

In order now to be able to obtain a clear 3D representation of the insert in a high-resolution and contrasty representation of its immediate vessel system environment, the 3D data set of the vessel system (e.g. pathogenic coronary vessel) computed in step S6 is combined or more precisely merged with the 3D data set of the insert computed in step S5. It should be noted here that usual 3D registration for image superimpositions is no longer necessary, as 2D matching of the two 3D image data sets has already been carried out according to step S4.

To ensure that during merging of the 3D data sets with and without contrast agent the 3D representation of the insert does not disappear due to merging with the 3D representation of the vessel anatomy, some of the 3D data sets are advantageously inverted and/or displayed with different colors.

Using the method according to steps S1 to S7 just described—in addition to improved visualization of vascular inserts—the position and attitude of one or more vascular inserts relative to the vessel anatomy can be made three-dimensionally visible and therefore the success of an implantation (e.g. a stent implantation) can be significantly better assessed than is possible with mere conventional two-dimensional visualization of vascular inserts according to the prior art.

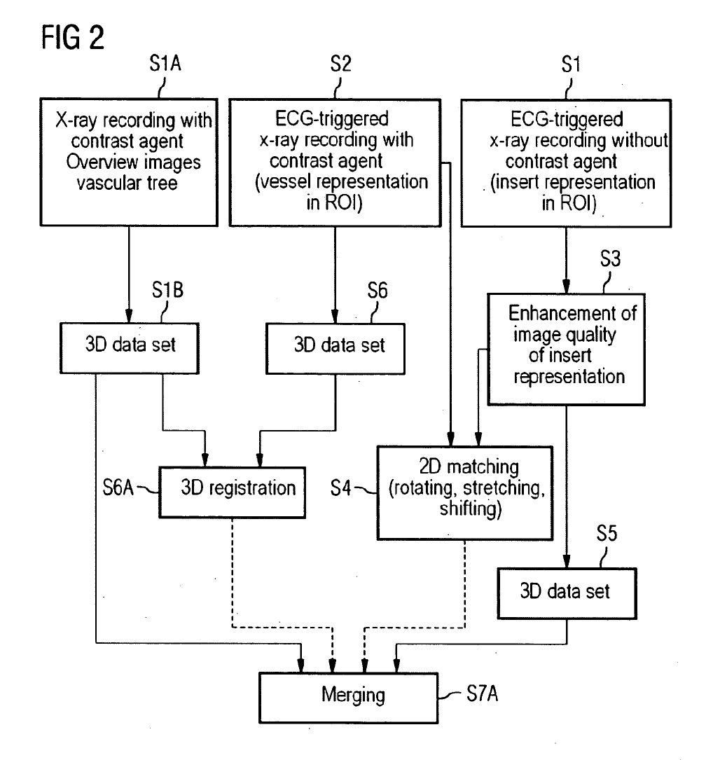

However, the described method according to the invention can be markedly improved still further in respect of the quality and quantity the 3D representation of the vessel system or vascular tree (FIG. 2).

For this purpose, in a step S1A (FIG. 2) [preceding] the ECG-triggered x-ray recording of the ROI acquired according to step S1, an x-ray recording (in the form of two or more x-ray projections at markedly different angles) of the entire vascular tree is made and, in a step S1B, there is computed therefrom a 3D data set which as such constitutes an overview image. Said 3D data set or rather this overview image is used to represent the anatomy of the entire vascular tree including the diseased vessel (internal lumen) and in particular the lesion to be treated using a vascular insert (e.g. stent).

In order to be able to correctly embed the high-resolution 3D data set of the insert with its ROI obtained according to step S5 in the (necessarily low-resolution) overview image of step S1B, the two 3D data sets (S1B, S5) must be three-dimensionally registered to one another. As 2D matching between the contrast-agent-based ROI recording (S2) and the enhanced insert representation (S3) has already been performed according to step S4, 3D registration (according to step S6A) is now only necessary between the overview image (3D data set S1B) and the [data set] according to step S6 from the contrast-agent-based ROI recording. A position sensor incorporated on or in the insert can advantageously be used for registration of the two 3D data sets.

On the basis of said 3D registration S6A and on the basis of the 2D matching S4, the high-resolution 3D data set of the ROI computed in S5 can then, in a step S7A, be superimposed on the 3D data set computed in S1B (overview image of the vascular tree).

If data acquisition according to step S1 A does not take place until after implantation of the vascular insert, a higher degree of consistency of the 2D recordings of the overall method is provided.

However, it may be advantageous to make additional (overview) recordings of the (diseased) vessel system even before implantation of an insert in order to reconstruct an additional 3D data set of the lesion to be treated. This 3D data set is also merged (possibly in a final step) with the other 3D data sets using 3D registration. In this way the conditions before and after implantation can be compared, thereby enabling the success of the treatment (e.g. stent implantation) to be even better verified.

It should be noted that as an alternative to steps S1A and S1B it is also possible to use a 3D data set representing the lesion to be-treated obtained from a CT or MRT recording made prior to the implantation.

1-10. (canceled)

11. A method for improving a 3D visualization of both a vascular insert and a vessel anatomy surrounding the insert in a patient using a radiography, comprising:
contrast-agent-free recording a first two series of x-ray images in different angulations of the vessel anatomy surrounding the vascular insert using an x-ray unit;
contrast-agent-based recording a second two series of x-ray images in the angulations of the vessel anatomy surrounding the vascular insert using the x-ray unit;
processing the first two series of x-ray images by a image processing method to improve an image quality of the insert;
matching the vessel anatomy from the second two series of x-ray images to the insert from the processed first two series of x-ray images by a 2D matching;
computing a first 3D image data set of a region enclosing the insert based on the processed first two series of x-ray images;
computing a second 3D image data set of the vessel anatomy based on the second two series of x-ray images; and
superimposing the second 3D image data set on the first 3D image data set based on the 2D matching.

12. The method as claimed in claim 11,
wherein a plurality of contrast-agent-based x-ray images of the vessel anatomy are recorded with a plurality of different x-ray projections in respect of a plurality of different projection angle,
wherein a further 3D image data set is computed based on the plurality of contrast-agent-based x-ray images,
wherein a 3D registration of the second 3D image data set with the further 3D image data set is performed,
wherein the first 3D image data set is superimposed on the further 3D image data set based on the 2D matching and the 3D registration.

13. The method as claimed in claim 12, wherein a 3D CT image or a 3D MRT image of the vessel anatomy which is recorded previously is used as the further 3D image data set.

14. The method as claimed in claim 12, wherein the 2D matching is performed using an ECG and a respiration signals of the patient recorded simultaneously with the recording of the x-ray images.

15. The method as claimed in claim 12, wherein a position sensor which is on or in the insert is used for the 3D registration.

16. The method as claimed in claim 11, wherein the angulations are rotated in 90°.

17. The method as claimed in claim 11, wherein the image processing method is a segmentation or a selection technique.

18. The method as claimed in claim 11, wherein the vascular insert is selected from the group consisting of: a stent, a bypass, and an artificial heart valve.

19. The method as claimed in claim 11, wherein the recordings of the first and second two series of x-ray images are triggered by an ECG or a blood pressure measurement.

20. The method as claimed in claim 11, wherein the matching is performed using an ECG and a respiration signals of the patient recorded simultaneously with the recordings of the first and second two series of x-ray images.

21. The method as claimed in claim 11, wherein the radiography is a C-arm x-ray system.

22. An apparatus for improving a 3D visualization of both a vascular insert and a vessel anatomy surrounding the insert in a patient, comprising:
an x-ray system for recording a first two series contrast-agent-free x-ray images in different angulations of the vessel anatomy surrounding the vascular insert and a second two series contrast-agent-based x-ray images in the angulations of the vessel anatomy surrounding the vascular insert; and
a computer having a computer program comprising:
a computer subroutine for processing the first two series of x-ray images by a image processing method to improve an image quality of the insert,
a computer subroutine for matching the vessel anatomy from the second two series of x-ray images to the insert from the processed first two series of x-ray images by a 2D matching,
a computer subroutine for computing a first 3D image data set of a region enclosing the insert based on the processed first two series of x-ray images,
a computer subroutine for computing a second 3D image data set of the vessel anatomy based on the second two series of x-ray images, and
a computer subroutine for superimposing the second 3D image data set on the first 3D image data set based on the 2D matching.

23. The apparatus as claimed in claim 22, wherein the recordings of the first and second two series of x-ray images are triggered by an ECG or a blood pressure measurement.

24. The apparatus as claimed in claim 22, wherein the matching is performed using an ECG and a respiration signals of the patient recorded simultaneously with the recordings of the first and second two series of x-ray images.

25. The apparatus as claimed in claim 22, wherein the angulations are rotated in 90°.
